(19) United States
(12) Patent Application Publication
(10) Pub. No.: US 2010/0198295 A1
(43) Pub. Date: Aug. 5, 2010

(54) PERFORMING EXTENDED CAPTURE DETECTION TEST AFTER DETECTING INADEQUATE CAPTURE

(75) Inventors: Todd Sheldon, North Oaks, MN (US); Lynn A. Davenport, Roseville, MN (US)

Correspondence Address: Medtronic, Inc., 710 Medtronic Parkway, Minneapolis, MN, 55432, US

(73) Assignee: Medtronic, Inc., Minneapolis, MN (US)

(21) Appl. No.: 12/362,905

(22) Filed: Jan. 30, 2009

(51) Int. Cl. A61N 1/37

(52) U.S. Cl. 607/28

(57) ABSTRACT

Techniques are described for performing an extended capture detection test after detecting inadequate capture during a first capture detection test. An example system includes an implantable medical device that delivers pacing pulses to a patient, that periodically performs a first capture detection test to detect capture or loss of capture of the pacing pulses, and that detects inadequate capture during the first capture detection test, wherein in response to detecting the inadequate capture, the implantable medical device performs a second capture detection test that is longer than the first test. The system also includes a programmer device that programs the implantable medical device and that retrieves data from the implantable medical device corresponding to the second capture detection test. The example system may conserve battery power and prevent loss of current by performing the extended capture detection test only after detection of inadequate capture during the first test.

### TECHNICAL FIELD

This disclosure relates to implantable medical devices, and more particularly, to implantable medical devices that deliver cardiac pacing.

### BACKGROUND

Cardiac pacing is delivered to patients to treat a wide variety of cardiac dysfunctions. Cardiac pacing is often delivered by an implantable medical device (IMD), which may also provide cardioversion or defibrillation, if needed. The IMD delivers such stimulation to the heart via electrodes located on one or more leads, which are typically intracardiac leads.

Patients with heart failure are often treated with cardiac resynchronization therapy (CRT). CRT is a form of cardiac pacing. In some examples, CRT involves delivery of pacing pulses to both ventricles to synchronize their contraction. In other examples, CRT to one ventricle, such as the left ventricle, to synchronize its contraction with that of the right.

At times, a cardiac pacing pulse may fail to capture the myocardium. For example, the electrode of the lead may have shifted or become entirely dislodged from an implant site. This is generally detrimental to the efficacy of cardiac pacing, but particularly so if the loss of capture occurs in the left ventricle during CRT. It is generally desirable that CRT be delivered and capture the myocardium for all or substantially all cardiac cycles. For patients with heart failure requiring CRT, lack of left-ventricular pacing, can worsen the patient's condition rather than improve the patient's condition.

Various methods exist for detecting loss of capture. In some examples, a first pair of electrodes delivers a pacing pulse, and a second pair of electrodes detects an electrical signal indicative of capture. In other examples, a device detects a mechanical contraction of the heart at the target site.

Performing a test to detect loss of capture may result in extra drain on a battery or other power source within an IMD. In some cases, a test to detect loss of capture is combined with a test to determine a threshold amplitude for pacing, which results in loss of capture during at least one cardiac cycle. Accordingly, IMDs typically perform such tests periodically for a certain duration of time, e.g., 20 to 30 seconds per day, rather than constantly test for loss of capture.

### SUMMARY

In general, this disclosure discusses techniques for monitoring to detect inadequate capture, e.g., loss of capture. Brief periodic capture detection tests may fail to detect intermittent loss of capture that occurs during the substantially longer periods between these tests. Such loss of capture may be due to periodic movement or dislodgment of a lead or changes in the myocardium, as examples, and may be more likely when the determined threshold amplitude for pacing pulses is at or near a maximum available from an IMD. Loss of LV capture during CRT may result in a patient's condition not improving or deteriorating. Without knowledge of the inadequate capture, a clinician may misinterpret the patient's condition as being indicative of the patient deriving no benefit from CRT, or the patient experiencing worsening heart failure.

According to the disclosure, when an IMD detects inadequate capture during a first capture detection test, the IMD switches to an extended capture detection mode. In one example, the IMD detects inadequate capture during a brief, periodic, e.g., 20 second, capture detection test and, in response to the detection of inadequate capture, the IMD begins an extended capture detection test, e.g., that lasts for a 24-hour time period. During the extended capture detection test, the IMD detects whether pacing pulses captured or failed to capture the myocardium. In some examples, the IMD maintains record of each capture and loss of capture detected during the extended capture detection test to provide a metric describing inadequate capture, e.g., a percent of capture or loss of capture, a raw number of losses of capture, an average number of losses of capture per time period, a number corresponding to a series of consecutive losses of capture, or other data or metrics regarding capture and/or loss of capture.

In one example, a method comprises periodically performing a first capture detection test having a first duration, detecting inadequate capture during the first capture detection test, and, in response to detecting the inadequate capture during the first capture detection test, performing a second capture detection test having a second duration, wherein the second duration is greater than the first duration. Performing the first and second capture detection tests according to the method comprises delivering cardiac pacing stimulation from an implantable medical device to a heart of a patient.

In another example, an implantable medical device comprises a signal generator that delivers pacing pulses to a heart of a patient, a control unit that periodically performs a first capture detection test having a first duration to detect inadequate capture of the heart by the pacing pulses, and a capture detection module that detects inadequate capture of the heart by the pacing pulses during the first capture detection test. In response to detecting the inadequate capture during the first capture detection test, the control unit performs a second capture detection test having a second duration to detect inadequate capture of the heart by the pacing pulses. The second duration is greater than the first duration.

In another example, a system comprises an implantable medical device and a computing device. The implantable medical device delivers pacing pulses to a heart a patient, that periodically performs a first capture detection test having a first duration to detect inadequate capture of the heart by the pacing pulses, and that detects inadequate capture during the first capture detection test, wherein in response to detecting the inadequate capture, the implantable medical device performs a second capture detection test having a second duration, wherein the second duration is greater than the first duration. The computing device retrieves data from the implantable medical device corresponding to the second capture detection test.

In another example, a system comprises means for periodically performing a first capture detection test having a first duration, means for detecting inadequate capture during the first capture detection test, and means for responding to the detection of the inadequate capture by performing a second capture detection test having a second duration, wherein the second duration is greater than the first duration.

### BRIEF DESCRIPTION OF DRAWINGS

FIG. 1 is a conceptual diagram illustrating an example system that provides cardiac pacing to a heart of a patient.

FIG. 2 is a conceptual diagram illustrating an implantable medical device and leads of the therapy system of FIG. 1 in greater detail.

FIG. 3 is a conceptual diagram illustrating another example of a therapy system provides cardiac pacing to a heart of a patient.

FIG. 4 is a block diagram illustrating an example configuration of an implantable medical device.

FIG. 5 is block diagram illustrating an example configuration of a programmer configured to communicate with an implantable medical device.

FIG. 6 is a block diagram illustrating an example system that includes an external device, such as a server, and one or more computing devices.

FIG. 7 is a flowchart illustrating an example method for performing a second, extended, capture detection test upon detection of loss of capture during a first capture detection test.

### DETAILED DESCRIPTION

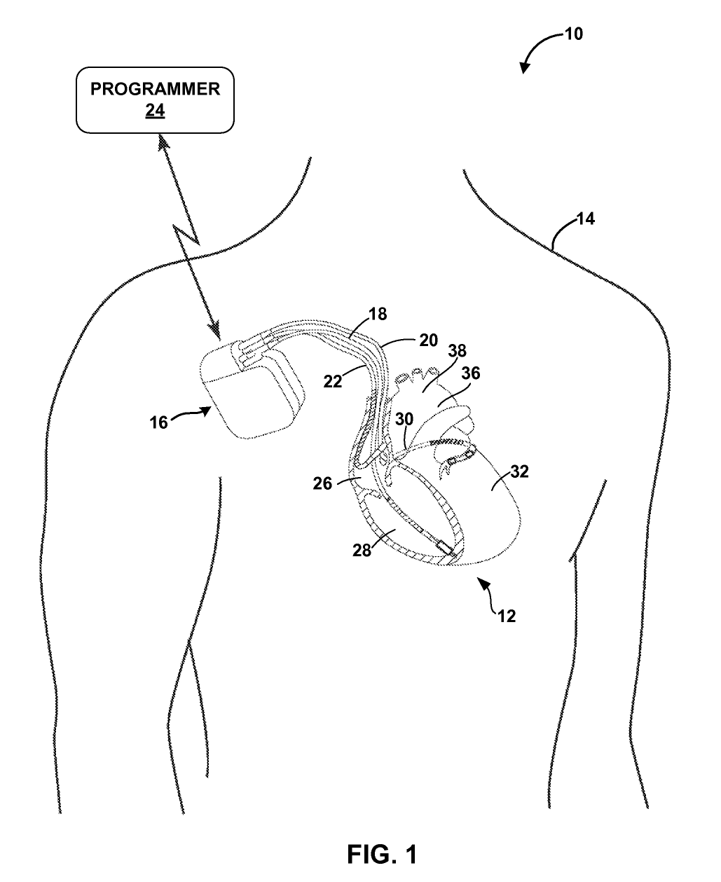

FIG. 1 is a conceptual diagram illustrating an example therapy system 10 that provides cardiac pacing therapy to a heart 12 of a patient 14. Therapy system 10 includes an IMD 16, which is coupled to leads 18, 20, and 22, and programmer 24. IMD 16 comprises a pacemaker, and may also comprise a cardioverter and/or defibrillator. IMD 16 provides pacing signals, and may also provide cardioversion or defibrillation signals, to heart 12 via electrodes coupled to one or more of leads 18, 20, and 22.

Leads 18, 20, 22 extend into the heart 12 of patient 16, and include electrodes (not shown) to sense electrical activity of heart 12 and deliver electrical stimulation to heart 12. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12.

In some examples, IMD 16 delivers pacing pulses to one or more the chambers of heart 12 based on the sensed electrical signals in such a manner as to provide cardiac resynchronization therapy (CRT) for patient 14. For CRT, IMD 16 delivers pacing pulses to the left ventricle, and may also deliver pacing pulses to the right ventricle, of heart 12. The delivery of pacing pulses to the ventricles may be timed from an intrinsic or paced depolarization of an atrium, e.g., the right atrium. In some examples, the delivery of a pacing pulse to the left ventricle is timed from an intrinsic or paced depolarization of the right ventricle.

IMD 16 periodically performs a first capture detection test having a first duration, and upon detecting inadequate capture, e.g., loss of capture, during the first capture detection test, IMD 16 performs a second capture detection test having a second duration that is greater than the first duration. For example, IMD 16 may perform the first capture detection test once per 24-hour period, e.g., for approximately 20 seconds, and upon detecting inadequate capture during the 20-second capture detection test, IMD 16 may begin a second capture detection test that lasts approximately 24 hours.

During the second capture detection test, IMD 16 records the results of the capture detection, e.g., whether IMD 16 detected capture or loss of capture. IMD 16 may also determine various statistics for capture and/or loss of capture detected during the second capture detection test. For example, IMD 16 may determine a number of losses of capture, a percentage for the number of captures or losses of capture relative to the number of delivered pacing pulses, a longest series of losses of capture, an average number of losses of capture over a period of time, or other statistics.

Programmer 24 may retrieve these statistics from IMD 16, or calculate these or other statistics from raw data gathered from IMD 16. Programmer 24 may further display the statistics and/or raw data to a user, e.g., via a user interface. For example, programmer 24 may generate and display a graph of a trend of capture and/or loss of capture over time, a graphical or textual representation of percent of capture or loss of capture, or a graphical or textual representation of a longest series of pulses for which IMD 16 detected loss of capture and the time at which this series occurred. As another example, programmer 24 may generate and display a histogram that presents a graphical representation of numbers of loss of capture events sorted by a duration, e.g., number of consecutive losses of capture in an event, or any other graphical or textual representations of loss of capture data.

IMD 16 may determine that inadequate capture has occurred during the first test according to various criteria. In one example, IMD 16 determines that inadequate capture occurs during the first capture detection test when any pacing pulse delivered during the first capture detection test fails to capture the myocardium. In another example, IMD 16 determines that inadequate capture occurs when each of a series of pacing pulses delivered during the first capture detection test fails to capture, e.g., a series of five pulses in a row fail to capture. In another example, IMD 16 determines that inadequate capture occurs when a threshold number of pacing pulses delivered during the first capture detection test fail to capture the myocardium. For example, for N pacing pulses delivered during the first capture detection test, IMD 16 may determine that inadequate capture occurs when M of the N pacing pulses fail to capture.

In some examples, IMD 16 performs the first capture detection test during a thresholding procedure to determine an amplitude or pulse width to apply for delivering pacing pulses. In some examples, IMD 16 delivers a series of pulses to the left ventricle of heart 12 while performing the first capture detection test. For each of the pulses in the series, when IMD 16 detects capture of the pulse, IMD 16 may decrease an applied amplitude (or pulse width) for a subsequent second pulse. IMD 16 may deliver the first pulse in the series at a relatively high amplitude and decrease the amplitude for each of the pulses by an amplitude step. IMD 16 may determine that loss of capture above a certain threshold, e.g., a threshold amplitude, corresponds to inadequate capture, as described below.

When IMD 16 detects loss of capture after delivering several pulses in the series, IMD 16 may set the pulse amplitude at a level corresponding to the amplitude applied when capture was last detected plus a safety margin to ensure that capture occurs during subsequent cardiac pacing. IMD 16 may then use the determined pulse amplitude for a period of time, e.g., 24 hours. In this manner, IMD 16 may deliver the pacing pulses at a voltage low enough to conserve battery power but high enough to ensure capture.

Under certain circumstances, during such a thresholding operation, IMD 16 detects loss of capture at a relatively high amplitude. For example, IMD 16 may detect loss of capture at the first pulse, e.g., a pulse delivered at the relatively high amplitude, or within several steps of the first pulse. IMD 16 may perform an extended capture detection test when IMD 16 detects loss of capture at the relatively high amplitude or width. The duration of the extended capture detection test may exceed the duration of the first capture detection test, e.g., the duration of the second capture detection test may last approximately 24 hours. In one example, when IMD 16 detects loss of capture before IMD 16 has reduced the amplitude below a maximum amplitude available from the IMD less the safety margin, IMD 16 determines that inadequate capture has occurred.

In some examples, IMD 16 detects inadequate capture when a plurality of capture thresholds determined during one or more thresholding procedures vary by greater than a threshold amount of variation. IMD 16 determines the variability of the capture thresholds using any of a variety of techniques, such as determining a difference between adjacent (in time) thresholds, a mean or median of such differences, or some other statistical calculation of variability. The determined variability value may be compared to a threshold to determine whether the variability is great enough for the IMD to detect inadequate capture.

In some examples, programmer 24 comprises a handheld computing device, computer workstation, or networked computing device. Programmer 24 includes a user interface that receives input from a user and presents information to the user. A user, such as a physician, technician, surgeon, electrophysiologist, or other clinician, may interact with programmer 24 to communicate with IMD 16. For example, the user may interact with programmer 24 to retrieve physiological or diagnostic information from IMD 16. A user may also interact with programmer 24 to program IMD 16, e.g., select values for operational parameters of the IMD.

The user may use programmer 24 to retrieve information from IMD 16 regarding detected capture and inadequate capture. For example, programmer 24 may retrieve data corresponding to whether IMD 16 determined that inadequate capture occurred during the first capture detection test. Programmer 24 may also retrieve recorded data corresponding to the number of times IMD 16 detected capture and/or inadequate capture during the second capture detection test. When IMD 16 records statistics, such as a percent inadequate capture, programmer 24 retrieves the recorded statistics from IMD 16. In one example, programmer 24 calculates and presents statistics from raw data retrieved from IMD 16, rather than IMD 16 calculating the statistics. For example, programmer 24 may calculate and present a percentage for the number of captures or number of inadequate captures, e.g., losses of capture, relative to the number of delivered pacing pulses. Furthermore, the user may define a duration for the first capture detection test, a frequency to perform the first capture detection test, a duration for the second capture detection test, inadequate capture that triggers the second capture detection test, or other parameters for the first and/or second capture detection tests using programmer 24.

In some examples, a user may program IMD 16 to vary the duration of the second capture test according to data gathered during the first capture detection test. For example, IMD 16 may establish the duration of the second capture detection test as a function of a percent of capture or loss of capture detected during the first capture detection test. As another example, when the first capture detection test corresponds to a thresholding procedure, IMD 16 may determine a duration for the second capture detection test based on a determined difference between a voltage at which IMD 16 detects inadequate capture and a threshold voltage. For example, the threshold voltage may be 2.5 volts, and IMD 16 may determine that, when inadequate capture is detected at 3.5 volts, IMD 16 will conduct the second capture detection test for 36 hours, but when inadequate capture is detected at 3 volts, IMD 16 will conduct the second capture detection test for 24 hours.

IMD 16 and programmer 24 may communicate via wireless communication using any techniques known in the art. In some examples, IMD 16 may include a response module that sends an alert to, e.g., programmer 24 when IMD 16 detects a problem with heart 12 or other organs or systems of patient 14. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

In one example, data regarding inadequate capture gathered via IMD 16 is presented with data regarding intrathoracic impedance measurements of patient 14, or other sensed data indicating the status of heart failure in the patient. IMD 16 may collect such data via electrodes on leads 18, 20 and 22, and provide the data to programmer 24. A user may utilize heart failure data in combination with inadequate capture data, for example, to determine effectiveness of a stimulation therapy administered to patient 14, e.g., by IMD 16. The user may also determine a change in the status of patient 14 by observing the heart failure data in combination with the inadequate capture data. In general, a user may utilize heart failure data in combination with inadequate capture data to identify the actual effectiveness of CRT and progression of heart failure in patient 14.

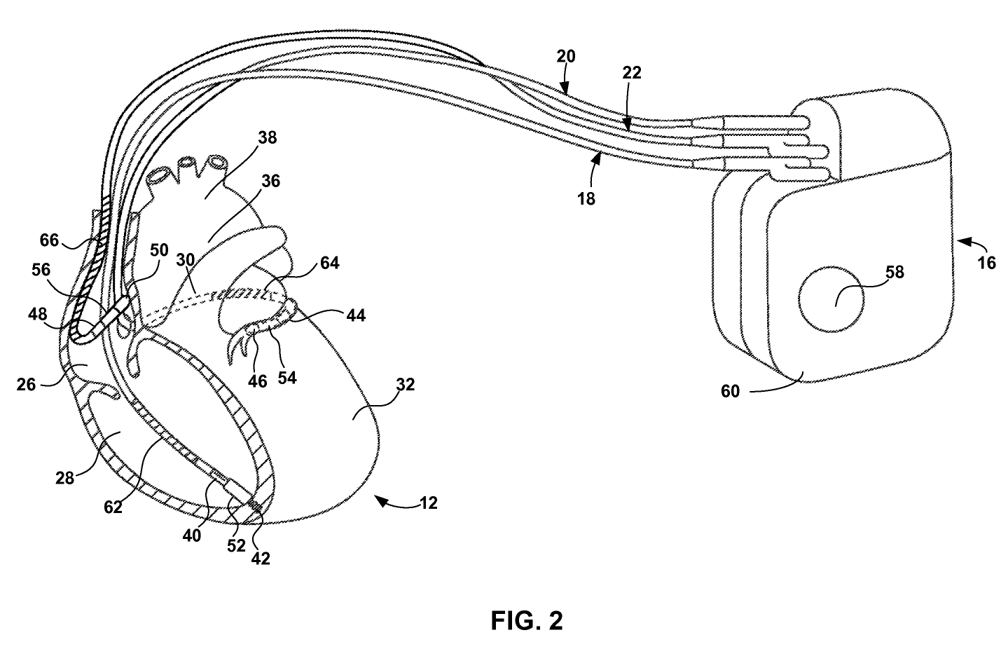

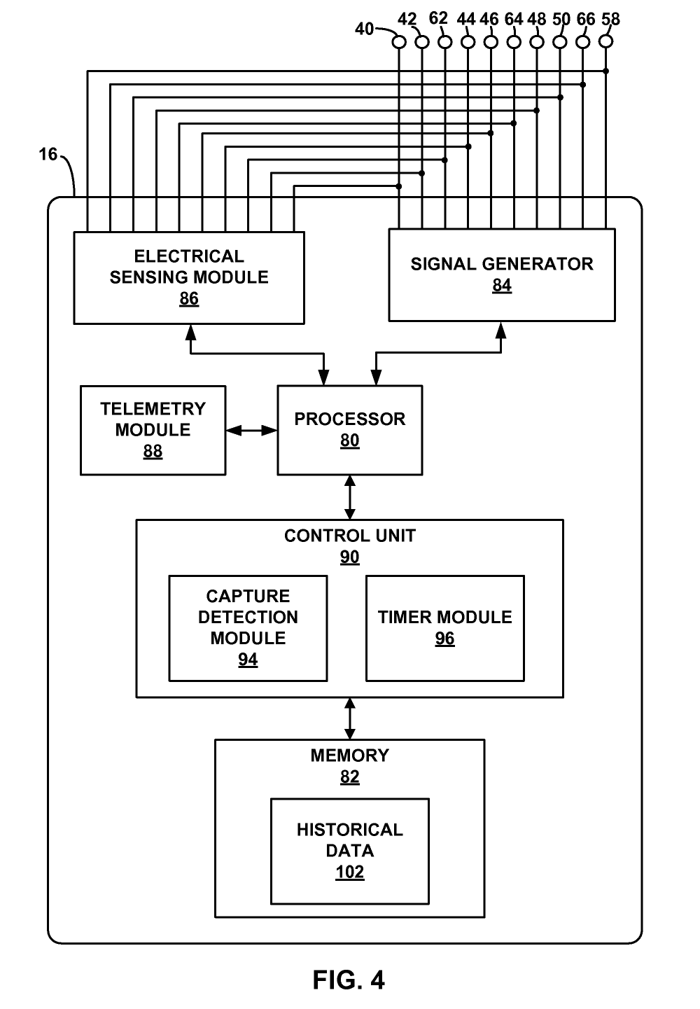

FIG. 2 is a conceptual diagram illustrating IMD 16 and leads 18, 20, 22 of therapy system 10 in greater detail. Leads 18, 20, 22 include conductors that are electrically coupled to a stimulation generator and a sensing module (FIG. 4) within a housing 60 of IMD 16. The conductors are coupled to electrodes on the leads.

Bipolar electrodes 40 and 42 are located adjacent to a distal end of lead 18 in right ventricle 28. In addition, bipolar electrodes 44 and 46 are located adjacent to a distal end of lead 20 in coronary sinus 30 and bipolar electrodes 48 and 50 are located adjacent to a distal end of lead 22 in right atrium 26. Leads 18, 20, 22 also include elongated electrodes 62, 64, 66, respectively, which may take the form of a coil. There are no electrodes located in left atrium 36, but other examples may include electrodes in left atrium 36. Furthermore, other examples may include electrodes in other locations, such as the aorta or a vena cava, or epicardial or extracardial electrodes proximate to any of the chambers or vessels described herein. Each of the electrodes 40, 42, 44, 46, 48, 50, 62, 64, and 66 may be electrically coupled to a respective conductor within the lead body of its associated lead 18, 20, 22, and thereby coupled to the stimulation generator and sensing module within housing 60 of IMD 16.

In some examples, as illustrated in FIG. 2, IMD 16 includes one or more housing electrodes, such as housing electrode 58, which may be formed integrally with an outer surface of hermetically-sealed housing 60 of IMD 16 or otherwise coupled to housing 60. In some examples, housing electrode 58 is defined by an uninsulated portion of an outward facing portion of housing 60 of IMD 16. Other division between insulated and uninsulated portions of housing 60 may be employed to define two or more housing electrodes. In some examples, housing electrode 58 comprises substantially all of housing 60. Housing electrode 58 is also coupled to one or both of the stimulation generator and sensing module within housing 60 of IMD 16.

IMD 16 senses electrical signals attendant to the depolarization and repolarization of heart 12 via any combination of electrodes 40, 42, 44, 46, 48, 50, 58, 62, 64, and 66. The electrical signals are conducted to IMD 16 from the electrodes via the respective leads 18, 20, 22 or, in the case of housing electrode 58, a conductor coupled to housing electrode 58. IMD 16 may sense such electrical signals via any bipolar combination of electrodes 40, 42, 44, 46, 48, 50, 58, 62, 64, and 66. Furthermore, any of the electrodes 40, 42, 44, 46, 48, 50, 58, 62, 64, and 66 may be used for unipolar sensing in combination with housing electrode 58.

In some examples, IMD 16 delivers pacing pulses via bipolar combinations of electrodes 40, 42, 44, 46, 48 and 50 to produce depolarization of cardiac tissue of heart 12. In some examples, IMD 16 delivers pacing pulses via any of electrodes 40, 42, 44, 46, 48 and 50 in combination with housing electrode 58 in a unipolar configuration. Furthermore, IMD 16 may deliver pacing pulses to heart 12 via any combination of elongated electrodes 62, 64, 66, and housing electrode 58. Electrodes 58, 62, 64, 66 may also be used to deliver cardioversion or defibrillation pulses to heart 12.

Any combination of electrodes 40, 42, 44, 46, 48, 50, 60, 62, 64 and 66 may be used for detecting capture or loss of capture in accordance with the techniques of this disclosure. In some examples, a first pair of electrodes is selected to deliver a pacing pulse and a second pair of electrodes is selected to detect capture of the myocardium by the pacing pulse delivered by the first pair of electrodes, e.g., by detecting the resulting depolarization of the myocardium and its timing relative to the pacing pulse. In some examples, IMD 16 detects loss of capture when a depolarization is not detecting within an interval that starts at the delivery of the pacing pulse. A later detected depolarization may be the result of condition from another chamber of heart 12, e.g., conduction from RV 28 to LV 32. For example, electrodes 42 and 46 may be used to detect capture or loss of capture for LV 32.

In other examples, IMD 16 detects capture or inadequate capture, e.g., loss of capture, by detecting mechanical contraction of heart 12 responsive to the pacing pulse, e.g., mechanical contraction of left ventricle 32. In such examples, IMD 16 may be coupled to a sensor that generates a signal that varies as a function of mechanical contraction of heart 12 via one of leads 18, 20 and 22, or another lead. Example sensors that generate a signal that varies as a function of mechanical contraction of heart 12 include accelerometers, or intracardiac or systemic pressure sensors.

The configuration of therapy system 10 illustrated in FIGS. 1 and 2 is merely one example. It should be understood that various other electrode and lead configurations for delivering stimulus and for detecting loss of capture are within the scope of this disclosure. For example, a therapy system may include epicardial leads and/or patch electrodes instead of or in addition to transvenous leads 18, 20, 22 illustrated in FIG. 1. Further, IMD 16 need not be implanted within patient 14. For examples in which IMD 16 is not implanted in patient 14, IMD 16 may deliver pacing pulses and other therapies to heart 12 via percutaneous leads that extend through the skin of patient 14 to a variety of positions within or outside of heart 12.

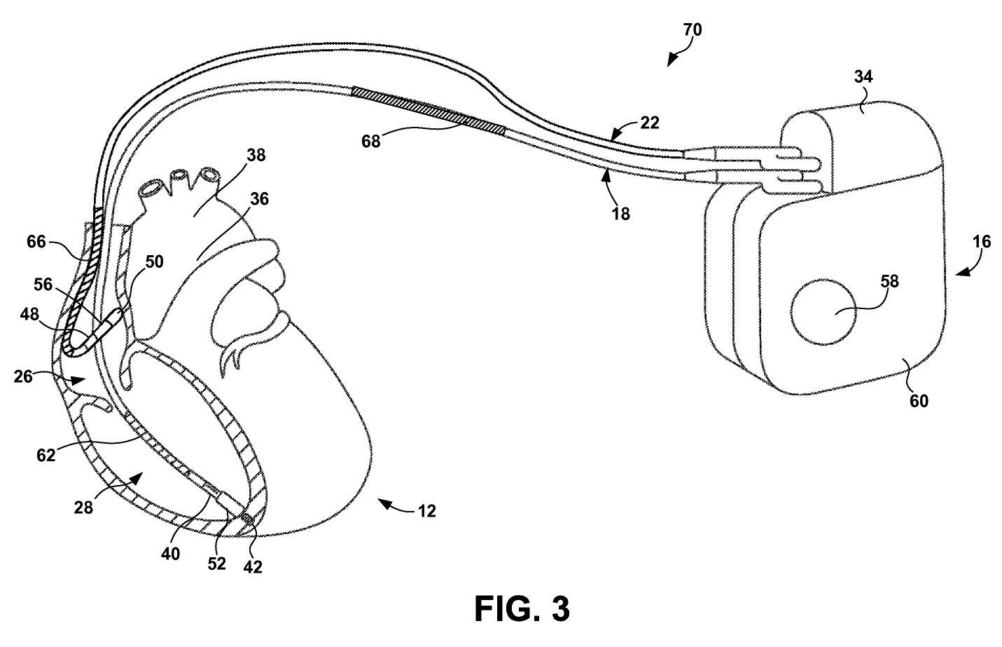

In addition, in other examples, a therapy system may include any suitable number of leads coupled to IMD 16, and each of the leads may extend to any location within or proximate to heart 12. For example, other examples of therapy systems may include three transvenous leads located as illustrated in FIGS. 1 and 2, and an additional lead located within or proximate to left atrium 36. As another example, other examples of therapy systems may include a single lead that extends from IMD 16 into right atrium 26 or right ventricle 28, or two leads that extend into a respective one of the right ventricle 26 and right atrium 26. An example of this type of therapy system is shown in FIG. 3. Any electrodes located on these additional leads may be used to detect capture or loss of capture during a first capture detection test and/or a second capture detection test, in accordance with the techniques described herein.

FIG. 3 is a conceptual diagram illustrating another example of therapy system 70, which is similar to therapy system 10 of FIGS. 1-2, but includes two leads 18, 22, rather than three leads. Leads 18, 22 are implanted within right ventricle 28 and right atrium 26, respectively. Additionally, lead 18 includes electrode 68, which may take the form of a coil, as in the example of FIG. 3. Therapy system 70 shown in FIG. 3 may also be useful for providing pacing pulses to heart 12. System 70 may also periodically perform a first capture detection test that lasts a first duration of time. Upon detecting inadequate capture during the first capture detection test, system 70 may perform a second capture detection test for an extended duration, i.e., longer than the first duration of the first capture detection test, in accordance with the techniques described herein. For example, system 70 may perform the first capture detection test for a duration of 20 seconds every 24-hour period, and upon detecting inadequate capture during the first capture detection test, system 70 may perform the second capture detection test for a duration of approximately one day.

FIG. 4 is a block diagram illustrating one example configuration of IMD 16. In the example illustrated by FIG. 4, IMD 16 includes a processor 80, memory 82, signal generator 84, electrical sensing module 86, and telemetry module 88. IMD 16 further includes control unit 90, which itself includes capture detection module 94 and timer module 96. Memory 82 may include computer-readable instructions that, when executed by processor 80, cause IMD 16 and processor 80 to perform various functions attributed to IMD 16, processor 80, or control unit 90 herein. The computer-readable instructions may be encoded within memory 82. Memory 82 may comprise any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Memory 82 also includes safety margin data 100 and historical data 102 in the example of FIG. 4.

Processor 80 and/or control unit 90 may include any one or more of a microprocessor, a controller, a digital signal processor (DSP), an application specific integrated circuit (ASIC), a field-programmable gate array (FPGA), or equivalent discrete or integrated logic circuitry. In some examples, processor 80 and/or control unit 90 may include multiple components, such as any combination of one or more microprocessors, one or more controllers, one or more DSPs, one or more ASICs, or one or more FPGAs, as well as other discrete or integrated logic circuitry. The functions attributed to processor 80 and/or control unit 90 herein may be embodied as software, firmware, hardware or any combination thereof. In one example, control unit 90, capture detection module 94, and timer module 96 may be stored or encoded as instructions in memory 82 that are executed by processor 80.

Processor 80 controls signal generator 84 to deliver stimulation therapy, e.g., cardiac pacing or CRT, to heart 12 according to a selected one or more therapy programs, which may be stored in memory 82. Signal generator 84 is electrically coupled to electrodes 40, 42, 44, 46, 48, 50, 58, 62, 64, and 66, e.g., via conductors of the respective lead 18, 20, 22, or, in the case of housing electrode 58, via an electrical conductor disposed within housing 60 of IMD 16. Signal generator 84 is configured to generate and deliver electrical stimulation therapy to heart 12 via selected combinations of electrodes 40, 42, 44, 46, 48, 50, 58, 62, 64, and 66. In some examples, signal generator 84 is configured to delivery cardiac pacing pulses. In other examples, signal generator 84 may deliver pacing or other types of stimulation in the form of other signals, such as sine waves, square waves, or other substantially continuous time signals.

Stimulation generator 84 may include a switch module and processor 80 may use the switch module to select, e.g., via a data/address bus, which of the available electrodes are used to deliver pacing pulses. Processor 80 may also control which of electrodes 40, 42, 44, 46, 48, 50, 58, 62, 64 and 66 is coupled to signal generator 84 for generating stimulus pulses, e.g., via the switch module. The switch module may include a switch array, switch matrix, multiplexer, or any other type of switching device suitable to selectively couple a signal to selected electrodes.

Electrical sensing module 86 monitors signals from at least one of electrodes 40, 42, 44, 46, 48, 50, 58, 62, 64 or 66 in order to monitor electrical activity of heart 12. Electrical sensing module 86 may also include a switch module to select which of the available electrodes are used to sense the cardiac activity. In some examples, processor 80 selects the electrodes that function as sense electrodes, or the sensing configuration, via the switch module within electrical sensing module 86.

Electrical sensing module 86 includes multiple detection channels, each of which may be selectively coupled to respective combinations of electrodes 40, 42, 44, 46, 48, 50, 58, 62, 64 or 66 to detect electrical activity of a particular chamber of heart 12. Each detection channel may comprise an amplifier that outputs an indication to processor 80 in response to detection of an event, such as a depolarization, in the respective chamber of heart 12. In this manner, processor 80 may detect the occurrence of R-waves and P-waves in the various chambers of heart 12.

Memory 82 stores intervals, counters, or other data used by processor 80 to control the delivery of pacing pulses by signal generator 84. Such data may include intervals and counters used by processor 80 to control the delivery pacing pulses to one or both of the left and right ventricles for CRT. The intervals and/or counters are, in some examples, used by processor 80 to control the timing of delivery of pacing pulses relative to an intrinsic or paced event, e.g., in another chamber.

In one example, capture detection module 94 uses electrical sensing module 86 to detect capture and/or inadequate capture when signal generator 84 delivers a pacing pulse. Via the switching module, processor 80 and/or capture detection module 94 may control which of electrodes 40, 42, 44, 46, 48, 50, 58, 62, 64 and 66 is coupled to electrical sensing module 86 to detect an invoked electrical response to a pacing pulse, i.e., capture. Memory 82 may store predetermined intervals or voltage thresholds which define whether a detected signal has an adequate magnitude and is appropriately timed relative to the pacing pulse to be considered an evoked response, i.e., capture. In some examples, a channel of electrical sensing module 86 used to detect capture comprises an amplifier which provides an indication to processor 80/capture detection module 96 when a detected signal has an adequate magnitude.

Processor 80 and/or control unit 90 control the selection of electrode configurations for delivering pacing pulses and for detecting capture and/or loss of capture. Processor 80, for example, may communicate with signal generator 84 to select two or more stimulation electrodes in order to generate one or more pacing pulses for delivery to a selected chamber of heart 12. Processor 80 may also communicate with electrical sensing module 86 to select two or more sensing electrodes for capture detection based on the chamber to which the pacing pulse is delivered by signal generator 84.

Control unit 90, in the example of FIG. 4, is capable of detecting inadequate capture during capture detection tests. In particular, in the example of FIG. 4, capture detection module 94 detects capture and/or loss capture during a first capture detection test. Control unit 90 uses timer module 96 to determine when to execute the first capture detection test, and for how long. For example, control unit 90 may initiate capture detection using capture detection module 94 when timer module 96 indicates that a time for performing a first capture detection test has been reached. Control unit 90 may also, in some examples, end the first capture detection test when timer module 96 indicates that a time for the first capture detection test, e.g., 20 seconds, has elapsed. In other examples, control unit ends the first capture detection test after a predetermined number of pacing pulses are delivered and evaluated, or after delivery of pacing pulses for a thresholding operation is complete.

Capture detection module 94 may determine inadequate capture during the first capture detection test according to various metrics. In one example, capture detection module 94 determines inadequate capture has occurred during the first capture detection test when any pacing pulse delivered during the first capture detection test fails to capture the myocardium. In another example, capture detection module 94 determines inadequate capture has occurred during the first capture detection test when a consecutive sequence of pacing pulses delivered during the first capture detection test, exceeding a minimum number of consecutive pulses, fail to capture the myocardium. In another example, capture detection module 94 determines inadequate capture has occurred during the first capture detection test when, for N pacing pulses delivered during the first capture detection test, M or more of the pacing pulses fail to capture the myocardium. In another example, capture detection module 94 determines inadequate capture has occurred during the first capture detection test when the variation between two or more capture thresholds exceeds a threshold value. In other examples, capture detection module 94 may determine inadequate capture during the first capture detection test using other metrics or a combination of metrics.

When capture detection module 94 detects inadequate capture during the first capture detection test, control unit 90 executes a second capture detection test having a duration greater than the duration of the first capture detection test. For example, control unit 90 may execute the second capture detection test for a period of approximately 24 hours, which control unit 90 may measure by referring to timer module 96. Capture detection module 94 may detect capture or loss of capture individually for substantially every pacing pulse delivered during the second capture detection test.

When capture detection module 94 detects loss of capture for a pacing pulse during the second capture detection test, control unit 90 may record the loss of capture in memory 82, e.g., as historical data 102. Historical data 102 may therefore comprise a record of losses of capture during the second capture detection test. In one example, control unit 90 may record an identifier for the second capture detection test that indicates that the second capture detection test was performed and uniquely identifies records of loss of capture detection as belonging to a particular second capture detection test, e.g., by recording a date on which the second capture detection test was performed, or a sequence number of the second capture detection test that increments each time control unit 90 executes the second capture detection test, or through other means. In some examples, control unit 90 similarly records each capture in memory 82, e.g., as historical data 102, instead of or in addition to recording each loss of capture.

In one example of a first capture detection test, control unit 90 determines an amplitude for pacing pulses according to a thresholding procedure. A voltage amplitude threshold is identified in the example thresholding procedure described below. In other examples, a current amplitude or pulse width threshold is determined using such a procedure. In any case, such a procedure may include or act as a first capture detection test.

For example, control unit 90 may cause signal generator 84 to deliver a first pacing pulse of a therapy period at a high voltage, e.g., Vₘₐₓ. Control unit 90 may further cause capture detection module 94 to detect capture or loss of capture of the first pacing pulse. When capture detection module 94 detects capture of the first pacing pulse, control unit 90 causes signal generator 84 to decrement the voltage by a voltage decrement, e.g., Vₛₜₑₚ, for the next pacing pulse. Control unit 90 causes voltage signal generator 84 to continue decrementing the delivered voltage by Vₛₜₑₚ for each consecutive pacing pulse until capture detection module 94 detects loss of capture for the pacing pulse. Control unit 90 then causes signal generator 82 to deliver subsequent pacing pulses at the last detected voltage plus a marginal increase in voltage as a safety margin to increase the likelihood of the pacing pulses capturing the myocardium.

In some examples, the time during which control unit 90 determines the voltage for the therapy period may correspond to the first capture detection test. During the first capture detection test, capture detection module 94 may detect loss of capture at Vₘₐₓ or within a specified number of steps of Vₘₐₓ. In one example, control unit 90 determines that inadequate capture occurs when control unit 90 detects that a pulse delivered within (Vₘₐₓ−safety-margin) fails to capture. Control unit 90 may respond by executing the second capture detection test. That is, control unit 90 may determine that failure to capture at Vₘₐₓ, or within a certain margin of Vₘₐₓ, is an inadequate capture that triggers the second capture detection test. Accordingly, control unit 90 may execute the second capture detection test, cause signal generator 84 to set the voltage at Vₘₐₓ, cause capture detection module 94 to detect capture and loss of capture during the second capture detection test, and record capture and/or loss of capture in historical data 102. Example pseudocode for the first capture detection test is presented below:

float First_Capture_Detection_Test (float VMax, float VStep, float safetyMargin, int
numLoss) {

| | |
|---|---|
| /* | First_Capture_Detection_Test returns a float value corresponding to a voltage |
| * | at which to deliver stimulation pulses based on detection of capture |
| * | VMax corresponds to a maximum output voltage |
| * | VStep corresponds to a step by which to decrement the voltage for each |
| * | detection of capture |
| * | safetyMargin corresponds to a voltage by which to increase a minimum |
| * | voltage at which capture is detected |
| * | numLoss corresponds to a number of steps within which to trigger the |
| * | Second_Capture_Detection_Test when loss of capture is detected |
| * | Detect_Capture( ) returns a Boolean value corresponding to whether capture is |
| * | detected when a pacing pulse is delivered at VCurrent |
| */ | |
| | float VCurrent = VMax; |
| | int capture = 0; |
| | while (Detect_Capture(VCurrent)) { |
| | VCurrent = VCurrent − VStep; |
| | capture = capture + 1; |
| | } |
| | if (capture < numLoss) { |
| | if (VCurrent + safetyMargin > VMax) |
| | VCurrent = VMax; |
| | else |
| | VCurrent = VCurrent + safetyMargin; |
| | Second_Capture_Detection_Test(VCurrent); |
| | return VCurrent; |
| | } |
| | else { |
| | VCurrent = VCurrent + safetyMargin; |
| | return VCurrent; |
| | } |

}

Although the example of IMD 16 of FIG. 4 includes capture detection module 94, in an alternative example, capture detection is performed by another device external to IMD 16. In one alternative example, a separate IMD detects capture or loss when IMD 16 delivers a pacing pulse. The separate IMD may detect capture, for example, by detecting the pacing pulse delivered by IMD 16, by detecting mechanical contraction of heart 12 in response to the pacing pulse, or through other means. As another example, a non-implanted device, such as an electrocardiograph or echocardiograph, may also detect capture or loss of capture when IMD 16 delivers a pacing pulse.

Telemetry module 88 includes any suitable hardware, firmware, software or any combination thereof for communicating with another device, such as programmer 24 (FIG. 1). Under the control of processor 80, telemetry module 88 may receive downlink telemetry from and send uplink telemetry to programmer 24 with the aid of an antenna, which may be internal and/or external. Processor 80 may provide data to be uplinked to programmer 24 and receive data from programmer 24 via telemetry module 88.

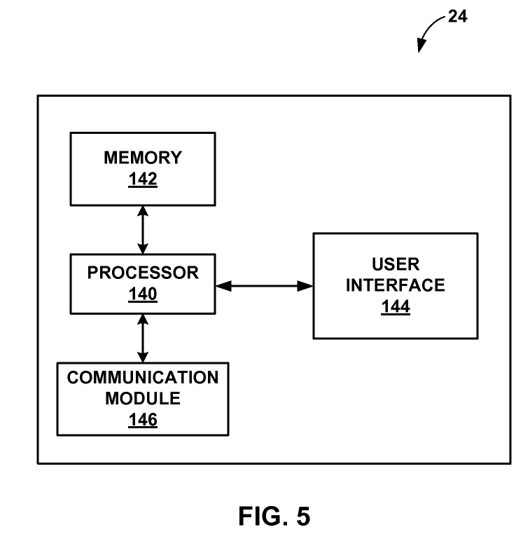

FIG. 5 is block diagram illustrating an example configuration of programmer 24. In general, a programmer may be a computing device. In the example shown in FIG. 5, programmer 24 includes a processor 140, memory 142, user interface 144, and communication module 146. Programmer 24 may be a dedicated hardware device with dedicated software for programming of IMD 16. Alternatively, programmer 24 may be an off-the-shelf computing device running an application that enables programmer 24 to program IMD 16. For example, programmer 24 may comprise a workstation computer, a laptop computer, a hand-held device such as a personal digital assistant (PDA), a cellular phone or smart phone, or other devices.

A clinician or other user interacts with programmer 24 via user interface 144, which may include a display to present a graphical user interface to a user, and a keypad, mouse, light pen, stylus, microphone for voice recognition, or other mechanism(s) for receiving input from a user. In some examples, processor 140 retrieves historical data 102 from IMD 16 via communication module 146, and controls user interface 144 to present graphical and/or textual representations of the data.

Processor 140 can take the form of one or more microprocessors, DSPs, ASICs, FPGAs, programmable logic circuitry, or the like, and the functions attributed to processor 140 herein may be embodied as hardware, firmware, software or any combination thereof. Memory 142 may store instructions that cause processor 140 to provide the functionality ascribed to programmer 24 herein, and information used by processor 140 to provide the functionality ascribed to programmer 24 herein. Additionally, processor 140 may perform the functionality of any or all of control unit 90, capture detection module 94, or timer module 96 described with respect to FIG. 4.

Memory 142 may include any fixed or removable magnetic, optical, or electrical media, such as RAM, ROM, CD-ROM, hard or floppy magnetic disks, EEPROM, or the like. Memory 142 may also include a removable memory portion that may be used to provide memory updates or increases in memory capacities. A removable memory may also allow patient data to be easily transferred to another computing device, or to be removed before programmer 24 is used to program therapy for another patient. Memory 142 may also store information that controls therapy delivery by IMD 16, such as stimulation parameter values.

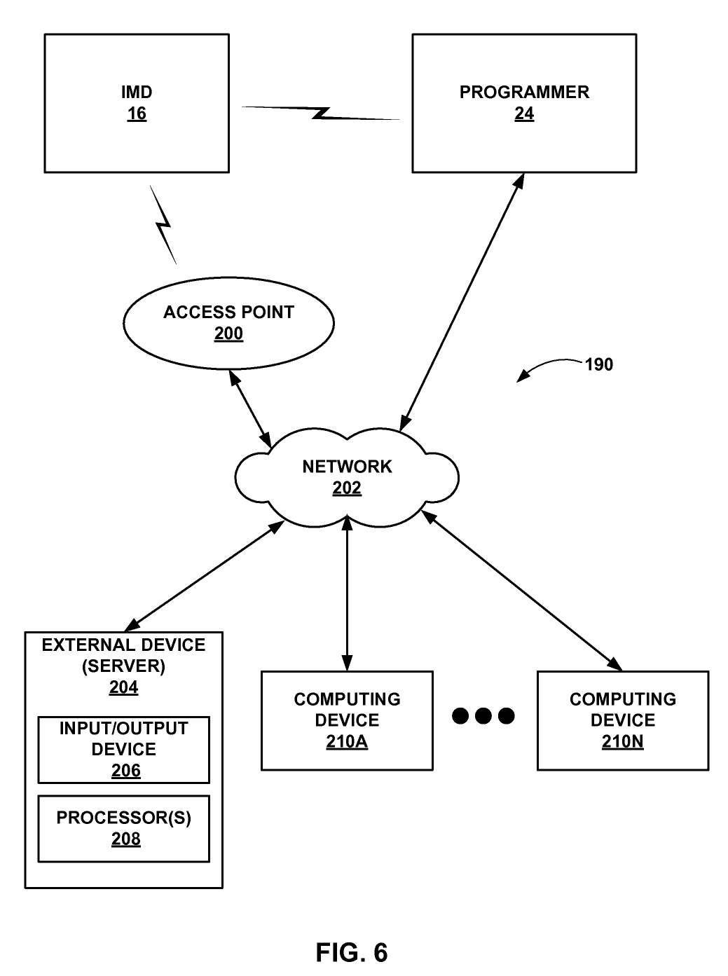

Programmer 24 may communicate wirelessly with IMD 16, such as by using RF communication or proximal inductive interaction. This wireless communication is possible through the use of communication module 146, which may be coupled to an internal antenna or an external antenna (not shown). Communication module 142 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection. An additional computing device in communication with programmer 24 may be a networked device such as a server capable of processing information retrieved from IMD 16. An example of such an arrangement is discussed with respect to FIG. 6.

Processor 140 of programmer 24 may implement any of the techniques described herein, or otherwise perform any of the methods described below. For example, processor 140 of programmer 24 may detect inadequate capture, record capture or loss of capture in memory 142, determine and record statistics regard capture or loss of capture, cause IMD 16 to execute a capture detection test, or other methods using any of the techniques described herein, e.g., based on measurements received from IMD 16 and/or commands received from a user or other entity. Processor 140 of programmer 24 may, in some examples, control the timing and configuration of the first and/or the second capture detection tests.

FIG. 6 is a block diagram illustrating an example system 190 that includes an external device, such as a server 204, and one or more computing devices 210A-210N (computing devices 210), that are coupled to IMD 16 and programmer 24 shown in FIG. 1 via a network 202. In this example, IMD 16 may use its telemetry module 88 to communicate with programmer 24 via a first wireless connection, and to communication with an access point 200 via a second wireless connection. In the example of FIG. 6, access point 200, programmer 24, server 204, and computing devices 210 are interconnected, and able to communicate with each other, through network 202. In some cases, one or more of access point 200, programmer 24, server 204, and computing devices 210 may be coupled to network 202 through one or more wireless connections. IMD 16, programmer 24, server 204, and computing devices 210 may each comprise one or more processors, such as one or more microprocessors, DSPs, ASICs, FPGAs, programmable logic circuitry, or the like, that may perform various functions and operations, such as those described herein.

Access point 200 may comprise a device that connects to network 186 via any of a variety of connections, such as telephone dial-up, digital subscriber line (DSL), fiber optic, wireless, or cable modem connections. In other examples, access point 200 may be coupled to network 202 through different forms of connections, including wired or wireless connections. In some examples, access point 200 may be co-located with patient 14 and may comprise one or more programming units and/or computing devices (e.g., one or more monitoring units) that may perform various functions and operations described herein. For example, access point 200 may include a home-monitoring unit that is co-located with patient 14 and that may monitor the activity of IMD 16.

In some examples, access point 200, server 204, or computing devices 210 may perform any of the various functions or operations described herein. For example, processor 208 of server 204 may detect inadequate capture during a first capture detection test and, upon detecting inadequate capture during the first capture detection test, execute a second capture detection test according to any of the techniques herein based on data received from IMD 16 via network 202. Processor 208 of server 204 may, in some examples, control the timing and configuration of stimulation pulses and capture detection by IMD 16 via network 202 and access point 200.

In some examples, IMD 16 may perform one or more additional actions upon detecting inadequate capture during a first and/or a second capture detection test. IMD 16 may, for example, send an alert to programmer 24, one or more of computing devices 210, server 204, or other device connected to network 202 when IMD 16 detects inadequate capture during either or both of the first capture detection test and the second capture detection test. As another example, IMD 16 may send a message to any device connected to network 202 that IMD 16 instructing a user, such as a clinician, that IMD 16 detected inadequate capture during the first and/or second capture detection test. IMD 16 may further be programmed to switch to a different combination of electrodes to deliver pacing pulses upon detecting inadequate capture during the second capture detection test.

In another example, IMD 16 may determine an action to take based on a number of inadequate captures, e.g., losses of capture, detected during the second capture detection test. For example, IMD 16 may send an alert when the number of inadequate captures is below a threshold during the second capture detection test, but IMD 16 may stop modify pacing therapy when the number of inadequate captures exceeds the threshold during the second capture detection test. IMD 16 may also use multiple thresholds to determine a responsive action, for example, a first threshold at which to send an alert and a second threshold at which to reconfigure electrodes.

In some cases, server 204 may be configured to provide a secure storage site for historical data 102 (FIG. 4) that has been collected from IMD 16 and/or programmer 24. Network 202 may comprise a local area network, wide area network, or global network, such as the Internet. In some cases, programmer 24 or server 204 may assemble historical data 102 in web pages or other documents for viewing by and trained professionals, such as clinicians, or by the patient, via viewing terminals associated with computing devices 210. Server 204 may also display the web pages or documents using input/output device 206. Processor 208 may also generate statistics regarding detected inadequate capture, e.g., an average number of inadequate captures, a median number of inadequate captures over a plurality of capture detection tests, a percentage of inadequate captures, a longest sequence of inadequate captures, voltages of stimulus pulses at which capture and/or inadequate capture was detected, a raw number of captures or losses of capture, or other statistics or measurements. The illustrated system of FIG. 6 may be implemented, in some aspects, with general network technology and functionality similar to that provided by the Medtronic CareLink® Network developed by Medtronic, Inc., of Minneapolis, Minn.

In one example, a user, such as a clinician, surgeon, physician, or other user, may remedy inadequate capture by adjusting a lead, or an electrode of a lead, for IMD 16 within patient 14, after reviewing output presented by programmer 24, server 204, computing devices 210, or other device in communication with IMD 16 regarding inadequate capture of IMD 16. The user may also attempt to non-invasively remedy the inadequate capture, e.g., by programming IMD 16 to use a different combination of electrodes to deliver pacing pulses.

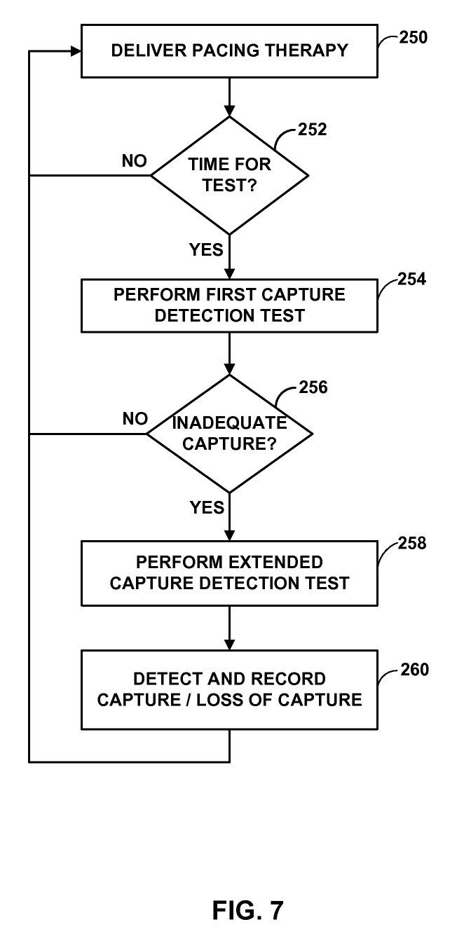

FIG. 7 is a flowchart illustrating an example method for executing a second capture detection test upon detection of inadequate capture during a first capture detection test. Although described with respect to IMD 16 of FIG. 1, it should be understood that any device, such as any combination of implantable or external devices described herein, may perform the example method of FIG. 7.

Initially, IMD 16 delivers pacing therapy, e.g., CRT, to heart 12 of patient 16 (250). Programmer 24 may download an initial pacing program to IMD 16 that includes parameters for the pacing program, such as an interval from a detected atrial event by which to deliver a ventricular pacing pulse, a voltage at which to deliver pacing pulses, a maximum voltage, a step by which to decrease the delivered voltage, times at which to initiate a first capture detection test, durations for a first capture detection test and a second capture detection test, a definition of inadequate capture that triggers a second capture detection test, or other parameters.

IMD 16 periodically determines whether a time to perform a first capture detection test has arrived (252). For example, timer module 96 (FIG. 4) may keep track of a time to perform the first capture detection test. If the time has not yet been reached (“NO” branch of 252), IMD 16 may continue to deliver the pacing therapy. However, when the time for the first capture detection test has been reached (“YES” branch of 252), IMD 16 performs the first capture detection test (254). The first capture detection test lasts for a relatively short duration, e.g., approximately 20 seconds. In general, the first capture detection test may correspond to any period of time during which IMD 16 tests for inadequate capture.

In one example, the first capture detection test (254) may correspond to a thresholding procedure used by IMD 16 to establish an amplitude or pulse width at which to deliver pacing pulses during a period of pacing therapy. The period may be, for example, a discrete time such as a day, a period of time between two programming events of IMD 16 by programmer 24, or other time period. During the thresholding procedure, IMD 16 determines whether a pacing pulse at a particular voltage, as one example, captures during the first capture detection test, and when the pacing pulse does capture, IMD 16 decrements the pacing pulse voltage by a specified voltage. IMD 16 continues decrementing the pacing pulse voltage until IMD 16 detects loss of capture for the pacing pulse, then uses a pacing pulse voltage at the last voltage at which capture was detected, plus a safety margin voltage. IMD 16 may determine that inadequate capture occurs when a pacing pulse fails to capture at an abnormally high voltage, e.g., at a maximum voltage or within several steps of the maximum voltage. That is, IMD 16 may receive from programmer 24 a value corresponding to a voltage above which, if IMD 16 detects capture loss for a pacing pulse delivered at the voltage, IMD 16 is to determine that inadequate capture has occurred during the first capture detection test for purposes of entering the second, extended capture detection test. In some examples, IMD 16 may further determine a duration for the second, extended capture detection test based on a difference between a voltage at which IMD 16 detected loss of capture and the value of the voltage received from programmer 24.

In some examples, the first capture detection test (254) may correspond to a periodic phase or mode of IMD 16 during which IMD 16 detects capture or inadequate capture. In one example, IMD 16 determines inadequate capture occurs (256) during the first capture detection test when any one of the pacing pulses delivered during the first capture detection test fails to capture. In another example, IMD 16 determines that inadequate capture occurs during the first capture detection test when each of a series of X pacing pulses delivered during the first capture detection test fails to capture, where X corresponds to a number of pacing pulses. In another example, IMD 16 delivers N pacing pulses during the first capture detection test and determines that inadequate capture occurs when M of N pacing pulses delivered during the first capture detection test fail to capture (M<N). That is, IMD 16 may determine that inadequate capture occurs when a percentage or a ratio of pulses delivered during the first capture detection test fail to capture. In another example, IMD 16 may determine that inadequate capture occurs during a thresholding procedure when IMD 16 detects loss of capture above a minimum voltage.

When IMD 16 does not detect inadequate capture (“NO” branch of 256), IMD 16 may continue to deliver pacing therapy according to the existing programmed parameters (250), without attempting to detect capture or inadequate capture further (e.g., to save battery power and to prevent loss of current for each pacing pulse) until the next time of a first capture detection test. When IMD 16 detects inadequate capture during the first capture detection test (“YES” branch of 256), e.g., because a pacing pulse at a relatively high voltage failed to capture or by detecting that one or more pacing pulses delivered during the first capture detection test failed to capture, IMD 16 performs an extended (e.g., second) capture detection test (258).

IMD 16 performs the extended capture detection test for a greater duration than the first capture detection test, e.g., for a 24-hour period. In one example, IMD 16 may establish the duration of the extended capture detection test during a thresholding procedure based on a difference between a voltage at which IMD 16 detects loss capture and a minimum voltage. For example, IMD 16 may use a first duration for a difference in one range and a second duration for a difference in another range. During the extended capture detection test, IMD 16 may detect whether each pacing pulse delivered captures or fails to capture. IMD 16 may also record whether each pacing pulse captures or fails to capture, e.g., in historical data 102 of memory 82 (FIG. 4) (260). IMD 16 may further calculate one or more statistics regarding capture or loss of capture, e.g., a percentage of pulses that captured or failed to capture, a longest series of pulses that failed to capture, an average number of pulses that failed to capture, or other statistics.

The techniques described herein may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described herein may also be embodied or encoded in a computer-readable medium, such as a computer-readable storage medium, containing instructions. Instructions embedded or encoded in a computer-readable medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

1. A method comprising:
periodically performing a first capture detection test having a first duration;
detecting inadequate capture during the first capture detection test; and
in response to detecting the inadequate capture during the first capture detection test, performing a second capture detection test having a second duration, wherein the second duration is greater than the first duration,
wherein performing the first and second capture detection tests comprises delivering cardiac pacing stimulation from an implantable medical device to a heart of a patient.

2. The method of claim 1, wherein the first capture detection test comprises a thresholding sequence for the cardiac pacing.

3. The method of claim 2, wherein detecting inadequate capture during the first capture detection test comprises detecting that a pacing pulse delivered from the implantable medical device at least one of at or within a range below a maximum amplitude of the implantable medical device failed to capture the heart.

4. The method of claim 1, wherein detecting inadequate capture during the first capture detection test comprises determining that a variability of capture thresholds exceeds a threshold.

5. The method of claim 1, wherein detecting inadequate capture during the first capture detection test comprises detecting that a pacing pulse delivered from the implantable medical device during the first capture detection test failed to capture the heart.

6. The method of claim 1, wherein detecting inadequate capture during the first capture detection test comprises detecting that a sequence of pacing pulses delivered from the implantable medical device during the first capture detection test failed to capture the heart.

7. The method of claim 1, wherein detecting inadequate capture during the first capture detection test comprises detecting that at least a percentage of pacing pulses delivered from the implantable medical device during the first capture detection test failed to capture the heart.

8. The method of claim 1, further comprising recording a number corresponding to at least one of pacing pulses delivered from the implantable medical device during the second capture detection test that failed to capture the heart or pacing pulses delivered from the implantable medical device during the second capture detection test that captured the heart.

9. The method of claim 8, further comprising determining a percentage of pacing pulses delivered from the implantable medical device during the second capture detection test that captured the heart.

10. The method of claim 9, further comprising presenting at least one of the number or the percentage to a user with a computing device that communicates with the implantable medical device.

11. The method of claim 1, wherein detecting inadequate capture during the first capture detection test comprises detecting inadequate capture with the implantable medical device.

12. An implantable medical device comprising:
a signal generator that delivers pacing pulses to a heart of a patient;
a control unit that periodically performs a first capture detection test having a first duration to detect inadequate capture of the heart by the pacing pulses; and
a capture detection module that detects inadequate capture of the heart by the pacing pulses during the first capture detection test,
wherein, in response to detecting the inadequate capture during the first capture detection test, the control unit performs a second capture detection test having a second duration to detect inadequate capture of the heart by the pacing pulses, wherein the second duration is greater than the first duration.

13. The device of claim 12, wherein the first capture detection test comprises a thresholding sequence, the signal generator delivers pacing pulses at a plurality of different amplitudes during the thresholding sequence, and the capture detection module at detects inadequate capture during the first capture detection test when a pacing pulse that is at least one of at or within a range below a maximum amplitude of the signal generator failed to capture the heart.

14. The device of claim 12, wherein the capture detection module detects inadequate capture during the first capture detection test when one of the pacing pulses delivered during the first capture detection test failed to capture the heart.

15. The device of claim 12, wherein the capture detection module detects inadequate capture during the first capture detection test when a sequence of pacing pulses delivered during the first capture detection test failed to capture the heart.

16. The device of claim 12, wherein the capture detection module detects inadequate capture during the first capture detection test when at least a percentage of pacing pulses delivered during the first capture detection test failed to capture the heart.

17. The device of claim 12, further comprising a memory, wherein the control module records at least one of a number corresponding to pacing pulses delivered during the second capture detection test that failed to capture the heart or a number corresponding to pacing pulses delivered during the second capture detection test that captured the heart within the memory.

18. The device of claim 17, wherein the control module determines a percentage of pacing pulses delivered during the second capture detection test that captured the heart.

19. A system comprising:
an implantable medical device that delivers pacing pulses to a heart a patient, that periodically performs a first capture detection test having a first duration to detect inadequate capture of the heart by the pacing pulses, and that detects inadequate capture during the first capture detection test, wherein in response to detecting the inadequate capture, the implantable medical device performs a second capture detection test having a second duration, wherein the second duration is greater than the first duration; and
a computing device that retrieves data from the implantable medical device corresponding to the second capture detection test.

20. The system of claim 19, wherein the first capture detection test comprises a thresholding sequence during which the implantable medical device determines an amplitude at which to deliver the pacing pulses.

21. The system of claim 20, wherein the implantable medical device detects inadequate capture during the first capture detection test when one of the pacing pulses that is at least one of at or within a range below a maximum amplitude of the implantable medical device failed to capture the heart.

22. The system of claim 19,
wherein the implantable medical device records at least one of a number corresponding to pacing pulses delivered during the second capture detection test that failed to capture the heart or a number corresponding to pacing pulses delivered during the second capture detection test that captured the heart, and transmits the at least one number to the computing device, and
wherein the computing device presents the at least one number to a user.

23. The system of claim 19, wherein at least one of the implantable medical device and the computing device determines a percentage of the pacing pulses delivered during the second capture detection test that captured the heart, and the computing device presents the percentage to a user.

24. The system of claim 19, wherein the computing device comprises programmer that programs the implantable medical device.

25. A system comprising:
means for periodically performing a first capture detection test having a first duration;
means for detecting inadequate capture during the first capture detection test; and
means for responding to the detection of the inadequate capture by performing a second capture detection test having a second duration, wherein the second duration is greater than the first duration.
